(19) United States
(12) Patent Application Publication
(10) Pub. No.: US 2005/0251233 A1
(43) Pub. Date: Nov. 10, 2005

(54) System and method for RF-induced hyperthermia

(76) Inventor: John Kanzius, Erie, PA (US)

Correspondence Address: CALFEE HALTER & GRISWOLD, LLP, 800 SUPERIOR AVENUE, SUITE 1400, CLEVELAND, OH, 44114, US

(21) Appl. No.: 10/969,477

(22) Filed: Oct. 20, 2004

Related U.S. Application Data
(60) Provisional application No. 60/569,348, filed on May 7, 2004.

(51) Int. Cl. A61F 2/00

(52) U.S. Cl. 607/101

(57) ABSTRACT

An embodiment of a non-invasive RF system for inducing hyperthermia in a target area, and a corresponding non-invasive RF method for inducing hyperthermia in a target area are provided. The system includes an RF transmitter and transmission head, and RF receiver and reception head wherein the transmission and reception heads are arranged proximate a target area so that an RF signal between the heads induces hyperthermia in the target area. The method includes arranging the transmission head and reception head proximate and on either side of a target area and transmitting an RF signal through the target area.

### CROSS REFERENCE TO RELATED APPLICATIONS

This application claims priority to, and the benefits of, provisional application Ser. No. 60/569,348 filed on May 7, 2004, which is also entitled System and Method For Rf-Induced Hyperthermia, and is incorporated herein by reference.

### FIELD OF THE INVENTION

The present invention relates generally to the field of radio frequency (RF) circuits, and more specifically to an RF transmitter and receiver system and method for inducing hyperthermia in a target area.

### BACKGROUND OF THE INVENTION

Hyperthermia is characterized by a very high fever, especially when induced artificially for therapeutic purposes. It is known in the art to use contact antennas to direct RF electromagnetic radiation to intentionally induce hyperthermia in human tissue for therapeutic purposes, e.g., destroying diseased cells. There are also RF heating devices known in the art (e.g., the Thermotron RF-8 system, Yamamoto Viniter Co. of Osaka, Japan, and the 3KCTPATEPM system, Russia).

When RF radiation is absorbed by matter it causes molecules to vibrate, which in turn causes heating. More specifically, RF waves interact with matter by causing molecules to oscillate with the electric field. This interaction has proven to be most effective for molecules that are polar, i.e. having their own internal electric field, such as water. Water molecules lose rotational energy via friction with other molecules, which causes an increase in temperature. This effect is the basis for microwave cooking. RF radiation absorbed by the body typically occurs as a result of the interaction of the RF radiation with water fluids contained in vivo.

The amount of RF radiation absorbed by tissue depends on a number of things, including the power and specific frequency of RF radiation used. Some frequencies of RF radiation have high absorption rates in tissue. A typical microwave oven emits RF radiation at about 2500 MHz, which is readily absorbed by water, fats and sugars to generate heat in food. RF radiation at lower frequencies, e.g., medium frequency (“MF”; 300 to 3000 kilohertz) RF radiation and high frequency (“HF”; 3 to 30 megahertz) RF radiation have generally low absorption rates in human tissue, even at relatively high powers, as evidenced by people safely standing near radio station tower transmitters, which transmit tens of thousands, and even hundreds of thousands, of Watts of RF power at lower frequencies.

RF ablation uses RF induced thermal energy to destroy tumor cells and involves placing a special needle into a tumor, often using image guidance. U.S. Pat. No. 4,800,899 discloses a system including a needle-like antenna that is inserted into a patient's body and into a tumor, permitting microwave RF energy supplied by a microwave generator to be applied directly to the tumor via the needle-like antenna to induce hyperthermia in the tumor. The RF energy generates heat in a volume (e.g., sphere) of tissue surrounding the needle. Ideally, the generated heat kills the tumor in a manner that spares the healthy tissue surrounding the tumor. RF ablation has several drawbacks, including the fact that treatment involves direct contact with the patient, i.e., insertion of a needle-like antenna into the patent for the duration of the procedure, which can require sedation and possibly an overnight stay in a hospital.

### SUMMARY OF THE INVENTION

In accordance with one embodiment of the present invention, a system for inducing hyperthermia in at least a portion of a target area is provided. The system includes an RF transmitter having an RF generator and a transmission head, and an RF receiver having a resonant circuit and a reception head. When the transmission and reception heads are arranged proximate to and on either side of a target area and an RF signal is transmitted from the transmission head, through the target area, to the reception head, at least a portion of the target area is warmed without direct contact of the heads to the target area.

In accordance with another embodiment of the present invention, a method of inducing hyperthermia in at least a portion of the target area is provided. The method includes providing an RF transmitter having an RF generator and a transmission head. The method further includes providing an RF receiver having a resonant circuit and a reception head. The transmission head and reception heads are insulated from the body part and arranged proximate to and on either side of a target area. An RF signal is transmitted via the transmission head to the reception head so that it passes through and warms at least a portion of the target area.

In accordance with another embodiment of the present invention, a methodology of inducing hyperthermia in a specific target portion of the target area of a patient's body is provided. This embodiment includes providing an RF transmitter, generator and transmission head. It also includes, providing an RF receiver, resonant circuit, and reception head wherein the reception head is selected in accordance with the shape and size of the target area. Once again, the transmission and reception heads are insulated from the body part and arranged proximate to and on either side of the body part containing the target area. The methodology further includes providing antibodies bound to an RF absorption enhancer and injecting the antibodies into the patient. Waiting for a period of time for the antibodies to bind to at least one type of cells within the target area and transmitting an RF signal from the transmission head to the reception head thereby warming the specific target portion of the target area of the body part.

In accordance with yet another embodiment of the present invention, a method of inducing hyperthermia in at least target cells of a patient is provided. The methodology includes providing an RF transmitter, generator and transmission head. It also includes providing an RF receiver, resonant circuit and reception head. Providing antibodies bound to an RF absorption enhancer and injecting the antibodies into the patient. Waiting for a period of time for the antibodies to bind to at least one type of cells within the target area and transmitting an RF signal from the transmission head to the reception head thereby warming the specific target area.

In accordance with still yet another embodiment of the present invention, a method of inducing hyperthermia in at least target cells of a patient is provided. The methodology includes extracting target cells from the patient. Providing an RF transmitter, generator and transmission head. It also includes providing an RF receiver, resonant circuit and reception head. Providing antibodies bound to an RF absorption enhancer and introducing the antibodies to the extracted target cells. Waiting for a period of time for the antibodies to bind to at least one of the extracted target cells and transmitting an RF signal from the transmission head to the reception head thereby warming the target cells.

In yet another embodiment of the present invention, a method of separating target cells of a patient is provided. The methodology includes removing material from the patient containing at least the target cells and providing antibodies bound to a magnetic material that bind to the target cells. The methodology further includes exposing the target cells to the antibodies bound to the magnetic material, waiting for the antibodies to bind to some of the target cells and providing a magnetic field to attract or repel some of the target cells to thereby separate the target cells.

### BRIEF DESCRIPTION OF THE DRAWINGS

FIG. 1 is an exemplary high-level block diagram of a non-invasive RF system for inducing hyperthermia in a target area;

FIG. 2 is an exemplary medium-level block diagram of an RF system for inducing hyperthermia in a target area;

FIGS. 3, 3A, 4, 5 and 6 are exemplary embodiments of transmission heads and reception heads on either side of a target areas;

FIG. 7 is an exemplary high-level flowchart of an embodiment of a RF methodology for inducing hyperthermia in a target area;

FIG. 8 is an exemplary medium level flow chart of an embodiment of an RF methodology for inducing hyperthermia in a target area;

FIG. 9 is an exemplary medium level flow chart of an embodiment of an RF methodology for inducing in-vitro hyperthermia in a target area; and

FIG. 10 is an exemplary medium level flow chart of an embodiment of an magnetic methodology for separating cells.

### DETAILED DESCRIPTION

In the accompanying drawings which are incorporated in and constitute a part of the specification, exemplary embodiments of the invention are illustrated, which, together with a general description of the invention given above, and the detailed description given below, serve to example principles of the invention.

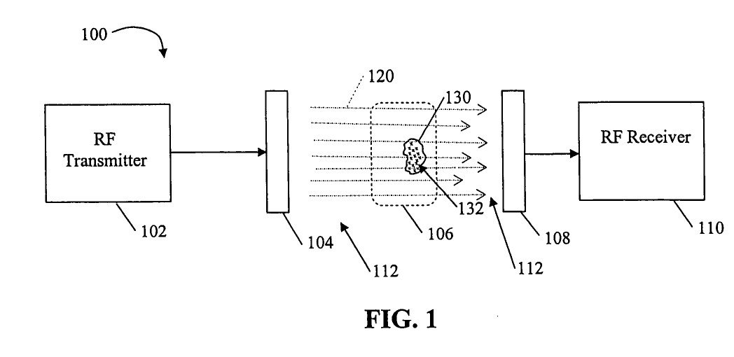

Referring to the drawings, and initially to FIG. 1, there is shown a first exemplary embodiment of a non-invasive RF system 100 for inducing hyperthermia in a target area 106. System 100 comprises an RF transmitter 102 in circuit communication with a transmission head 104 and an RF receiver 110 in circuit communication with a reception head 108. “Circuit communication” as used herein is used to indicate a communicative relationship between devices. Direct electrical, optical, and electromagnetic connections and indirect electrical, optical, and electromagnetic connections are examples of circuit communication. Two devices are in circuit communication if a signal from one is received by the other, regardless of whether the signal is modified by some other device. For example, two devices separated by one or more of the following—transformers, optoisolators, digital or analog buffers, analog integrators, other electronic circuitry, fiber optic transceivers, or even satellites—are in circuit communication if a signal from one reaches the other, even though the signal is modified by the intermediate device(s). As a final example, two devices not directly connected to each other (e.g. keyboard and memory), but both capable of interfacing with a third device, (e.g., a CPU), are in circuit communication.

In exemplary system 100, the RF transmitter 102 generates an RF signal 120 at a frequency for transmission via the transmission head 104. Optionally, the RF transmitter 102 has controls for adjusting the frequency and/or power of the generated RF signal and/or may have a mode in which an RF signal at a predetermined frequency and power are transmitted via transmission head 104. In addition, optionally, the RF transmitter 102 provides an RF signal with variable amplitudes, pulsed amplitudes, multiple frequencies, etc.

The RF receiver 110 is in circuit communication with the reception head 108. The RF receiver 110 is tuned so that at least a portion of the reception head 104 is resonant at the frequency of the RF signal 120 transmitted via the transmission head 104. As a result, the reception head 108 receives the RF signal 120 that is transmitted via the transmission head 104.

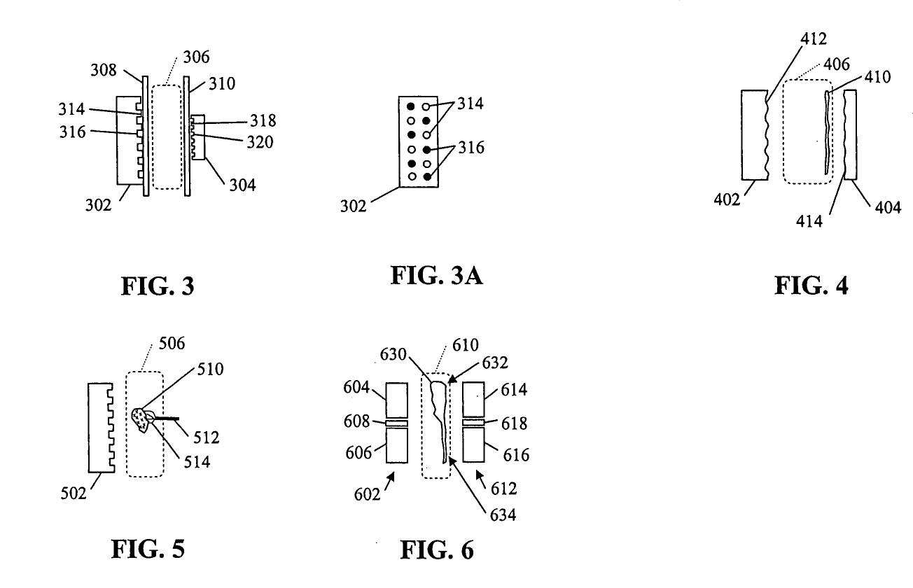

The transmission head 104 and reception head 108 are arranged proximate to and on either side of a general target area 106. General target 106 is general location of the area to be treated. The general target area 106 is any target area or type of cells or group of cells, such as for example, tissue, blood cells, bone marrow cells, etc. The transmission head 104 and reception head 108 are preferably insulated from direct contact with the general target area 106. Preferably, the transmission head 104 and reception head 108 are insulated by means of an air gap 112. Optional means of insulating the transmission head 104 and reception head 108 from the general target area 106 include inserting an insulating layer or material 310 (FIG. 3), such as, for example, Teflon® between the heads 104, 108 and the general target area 106. Other optional means include providing an insulation area on the heads 104, 108, allowing the heads to be put in direct contact with the general target area 106. The transmission head 104 and the reception head 108, described in more detail below, may include one or more plates of electrically conductive material.

The general target area 106 absorbs energy and is warmed as the RF signal 120 travels through the general target area 106. The more energy that is absorbed by an area, the higher the temperature increase in the area. Generally, the general target area 106 includes a specific target area 130. Specific target area 130 includes the tissue or higher concentration of cells, such as, for example, a tumor, that are desired to be treated by inducing hyperthermia. Preferably, the general target area is heated to for example, to between 106° and 107°. Thus, preferably, the specific target area 130 receives higher concentrations of the RF signal 120 then the general target area 106. As a result, the specific target area 130 absorbs more energy, resulting in a higher temperature in the specific target area 130 than in the surrounding general target area 106.

Energy absorption in a target area can be increased by increasing the RF signal 120 strength, which increases the amount of energy traveling through the general target area 106. Other means of increasing the energy absorption include concentrating the signal on a localized area, or specific target area 130, and/or enhancing the energy absorption characteristics of the target area 130.

One method of inducing a higher temperature in the specific target area 130 includes using a reception head that is smaller than the transmission head. The smaller reception head picks up more energy due to the use of a high-Q resonant circuit described in more detail below. Optionally, an RF absorption enhancer 132 is used. An RF absorption enhancer is any means or method of increasing the tendency of the specific target area 130 to absorb more energy from the RF signal. Injecting an aqueous solution is a means for enhancing RF absorption. Aqueous solutions suitable for enhancing RF absorption include, for example, water, saline solution, aqueous solutions containing suspended particles of electrically conductive material, such as metals, iron, various combination of metals, irons and metals, or magnetic particles. These types of RF enhancers are generally directly introduced to the target area. Preferably, these types of RF enhancers are directly injected into the target area by means of a needle and syringe.

Other means of enhancing RF absorption include providing antibodies with associated RF absorption enhancers, such as metal particles. The antibodies target and bind to specific target cells in the target area 130. Generally, antibodies can be directed against any target, e.g., tumor, bacterial, fungal, viral, parasitic, mycoplasmal, hisocampatabiltiy, differentiation and other cell membrane antigens, pathogen surface antigens, toxins, enzymes, allergens, drugs and any biologically active molecules. Binding RF enhancers to the antibodies permits the injection of the antibodies into the patient and the targeting of specific cells. Once a high enough concentration of RF enhancers 132 are attached to the target cells, the RF signal 120 is passed through the specific target area 130. The RF enhancers induce the absorption of more energy, creating a localized temperature in the specific target area 130 that is higher than the temperature created in the general target area 106. In addition, a combination of antibodies bound to different metals can be used allowing for variations in the RF absorption characteristics in localized areas of the target areas. These variations in RF absorption characteristics permit intentional uneven heating of the specific target area 130.

Doping or bonding antibodies with RF enhancers can be used to improve current RF capacitive heating devices as well as current RF ablation. Antibodies bound to metals RF absorption enhancers can be obtained through commercially available channels. One such commercial channel includes, for example, Research Diagnostics, Inc. located at Pleasant Hill Road, Flanders N.J.

The antibodies bond to RF enhancers are applicable for both in-vivo and in-vitro applications. In one in-vitro application the antibodies and RF enhancers are in introduced to the target area prior to the target area being removed from the patient. After the antibodies bind to the target area, the target area is removed from the patient and treated with one or more RF signals. In another in-vitro application the target area is removed from the patient before the RF enhancers are introduced to the target area. Once the target area is in a suitable vessel, the antibodies and RF enhancers are introduced to the target area. The target area is then treated with one or more RF signals.

Optionally, multiple frequency RF signals 120 are used. Multiple frequency RF signals can be used to treat target areas. Multiple frequency RF signals allow the energy absorption rate and absorption rate in different locations of the target area to be more closely controlled. The multiple frequency signals can be combined into one signal, or by use of a multi-plated transmission head, or multiple transmission heads, can be directed at one or more specific regions in the target area. This is useful for treating target areas that have specific regions of various shapes, thicknesses and/or depths. Similarly, pulsed RF signals, variable frequency RF signals and other combinations or variations of the RF signals can be used to more precisely control and target the heating of the specific target areas. These and other methods of increasing RF absorption can be used independently or in any number of combinations to increase the energy absorption rate of the specific target area 130.

In addition, antibodies bound with magnetic particles can be steered to specific locations using magnets or magnetic resonant imaging (MRI) machines. Thus, the antibodies can be directed toward specific target area or target cells. Furthermore, once the antibodies bind to the specific target cells, the target cells can be separated from the other cells by use of a magnetic force. The magnetic force can be either an attracting force, or a repelling force. Magnets, or MRI machines can also be used to steer injected magnetic particles to specific locations.

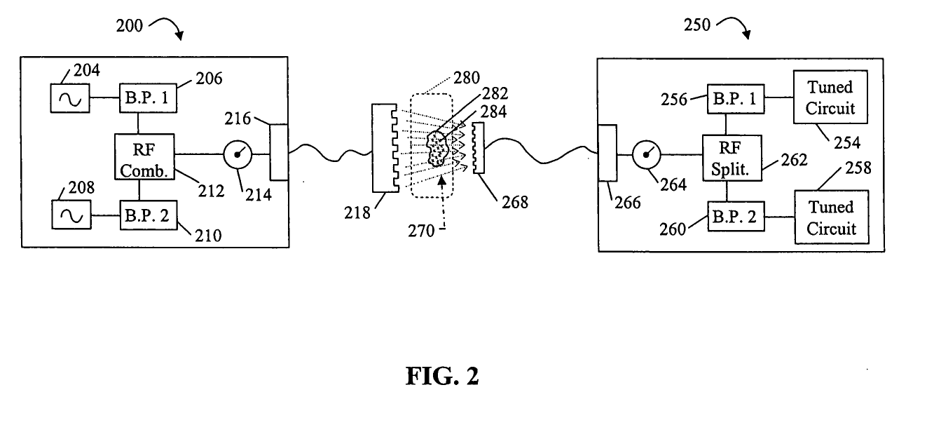

FIG. 2 illustrates an exemplary embodiment having an RF transmitter 200 in circuit communication with transmission head 218 that transmits an RF signal 270 through a target area 280 to a reception head 268 in circuit communication with an RF receiver 250. The RF transmitter 200 is a multi-frequency transmitter and includes a first RF signal generator 204. The first RF signal generator 204 generates a first signal at a first frequency F1, such as a 16 megahertz frequency. The first RF signal generator 204 is in circuit communications with band pass filter B.P. 1 206, which is in circuit communication with an RF combination circuit 212. Band pass filter B.P. 1 206 is a unidirectional band pass filter that prevents signals at other frequencies from reaching first RF signal generator 204.

RF transmitter 200 includes a second RF signal generator 208. Second RF signal generator 208 generates a second signal at a second frequency F2, such as, for example a 6 megahertz signal. Second signal generator 208 is in circuit communication with band pass filter B.P. 2 210, which is also in circuit communication with the RF combination circuit 212. Band pass filter B.P. 2 210 prevents signals at other frequencies from reaching second RF signal generator 208. Optionally, RF combination circuit 212 includes circuitry to prevent the first and second signals from flowing toward the other signal generators and thus eliminates the need for band pass filter B.P. 1 206 and band pass filter B.P. 2 210.

RF combination circuit 212 combines the first and second signal at frequency F1 and frequency F2 and outputs RF signal 270. Preferably, RF combination circuit 212 is in circuit communication with first meter 214. First meter 214 is used to detect the signal strength of RF signal 270. The RF signal 270 is transmitted via transmission head 218 through the target 280 to reception head 268. Optionally, plug type connectors 216, 266 are provided allowing for easy connection/disconnection of transmission head 218, and reception head 268 respectfully. Reception head 268 is preferably in circuit communications with a second meter 264. Second meter 264 detects the RF signal strength received by the reception head 268. The difference in RF signal strength between first meter 214 and second meter 264 can be used to calculate energy absorbed by the target area 280. Reception head 268 is also in circuit communication with an RF splitter 262. RF splitter 262 separates the RF signal 270 into back into its components, first signal at frequency F1 and second signal at frequency F2. RF splitter 262 is in circuit communication with band pass filter B.P. 1 256, which is in circuit communication with first tuned circuit 254. Similarly, RF splitter 262 is in circuit communication with band pass filter B.P. 2 260, which is in circuit communication with second tuned circuit 258. Optionally, band pass filter B.P. 1, 256 and band pass filter B.P. 2 260 can be replaced with a splitter or powered tee.

First tuned circuit 254 is tuned so that at least a portion of reception head 268 is resonant at frequency F1. Similarly, second tuned circuit 258 is tuned to that at least a portion of reception head 268 is resonant at frequency F2. Since the reception head 268 is resonant at frequencies F1 and F2 the RF signal 270 is forced to pass through the target area 280.

Optionally, an exemplary embodiment having an RF transmitter, similar to that illustrated above, that does not include an RF combination circuit is provided. Instead, the RF transmitter uses a multi-frequency transmission head. In this embodiment, one portion of the transmission head is used to transmit one frequency signal, and a second portion is used to transmit a second frequency signal. In addition, optionally, the reception head and resonant circuits are constructed without the need for a splitter, by providing a reception head having multiple portions wherein the specific portions are tuned to receive specific frequency signals. An example of such a transmission head in more detail illustrated below.

FIG. 2 illustrates another means for concentrating the RF signal on specific target area by using a larger transmission head then reception head. The RF signal 270 transmitted by larger transmission head 218 is received by reception head 268 in such a manner that the RF signal 270 is more concentrated near the reception head 268 than it is near the transmission head 218. The more concentrated the RF signal 270, the higher the amount of energy that can be absorbed by the specific area 282. Thus, positioning the larger transmission head on one side of the target area 280 and positioning the smaller reception head 268 on the other side of and near the specific target area 282 is a means for concentrating the RF signal 270 on the specific target area 282. Optionally, one or more of the tuned circuits 254, 258 in the RF receiver 250 are tuned to have a high quality factor or high “Q.” Providing a resonant circuit with a high “Q” allows the tuned head to pick up larger amounts of energy.

FIGS. 3-6 illustrate a number of exemplary transmitter head and reception head configurations. Additionally, the transmitter and receiver heads may be metallic plates. FIG. 3 illustrates a transmitter head 302 having a non-uniform thickness 314. Transmission head 302 is electrically insulated from target area 306 by an insulation layer 308 in contact with the target area. Similarly, reception head 304 is electrically insulated by insulation layer 310. Insulation layer 310 can be in direct contact with target area 306. Insulation layer 308, 310 provide additional means of electrically insulating the transmission head and reception heads from the target area. Reception head 304 also has non-uniform thicknesses 314 and 316. Receiver head 304 is smaller than transmission head 302 and has a smaller cross sectional area on its face. The smaller cross-sectional area of receiver head 304 facilitates in concentrating an RF signal in a specific target area.

FIG. 3A illustrates a face view of the exemplary embodiment of the transmission head 302 of FIG. 3. The transmission head 302 includes a plurality of individual transmission heads 314, 316. Transmission heads 314 provide for transmission of a signal at a first frequency, such as 4 megahertz. Transmission heads 316 provide for transmission of a signal at a second frequency, such as, for example 10 megahertz. Preferably, the transmission heads 314 and 316 are electrically insulated from one another. In addition, preferable the power output can be controlled to each transmission head, allowing for the power output to be increased or decreased in specific areas based upon the size, shape, or depth of the specific target area. Optionally, all of the transmission heads 314 provide the same power output, and transmission heads 316 provide the same power output.

Obviously the transmission head can contain any number of individual transmission heads. Moreover, the transmission heads can transmit signals at a plurality of frequency, and include, but are not limited to transmission heads that transmit signals at one, two, three, etc. different frequencies. All of which have been contemplated and are within the spirit and scope of the present invention.

FIG. 4 illustrates yet an additional exemplary embodiment. FIG. 4 illustrates transmission head 402 with a wavy surface 412 and reception head 404 having a wavy surface 414. Other useful surface configurations include bumpy, planer, non-uniform, mounded, conical and dimpled surfaces. Varied surface shapes allow for variable depths of heating control. The shape of receiving head 414 is thinner, narrower (not shown) and is selected based upon the size and shape of the specific target area 410 located in the general target area 406.

FIG. 5 illustrates an exemplary embodiment with a non-invasive transmission head 502 and an invasive needle 512. In this embodiment, end of needle 512 is located at least partially within general target area 506 and near specific target area 510. Needle 512 is preferably hollow and has extension members 514 within the needle 512. Once the end of needle 512 is located near the specific target area 510, the extension members 514 are extended and attach to the specific target area 510. Preferably, the specific target area 510 has been targeted with a large concentration of RF absorption enhancers 516. The target area 510, itself, becomes the reception head. The extension members 514 provide circuit communication with the resonant circuit and the target area 510 is resonant at the desired frequency. Providing multiple extension members provides for a more even heating of the specific target area 510. This embodiment allows the RF signal to be concentrated on small areas.

FIG. 6 illustrates yet another exemplary embodiment of transmission and reception heads. In this embodiment, transmission head 602 includes a first transmission head portion 604 and a second transmission head portion 606. The first and second transmission heads 602, 604 are electrically isolated from one another by an insulating member 608. Similarly, reception head 612 includes a first reception portion 614 and a second reception portion 16 that are electrically isolated from one another by an insulation member 618. Providing multiple transmission head portions that are electrically isolated from one another allows the use of multiple frequencies which can be used to heat various shapes and sizes of target areas. Different frequencies can be used to heat thicker and thinner portions of the target area, or deeper target areas allowing for a more uniform heating, or maximum desired heating, of the entire target area. Another exemplary embodiment (not shown) includes a plurality of concentric circles forming transmission head portions and are electrically isolated or insulated from each other.

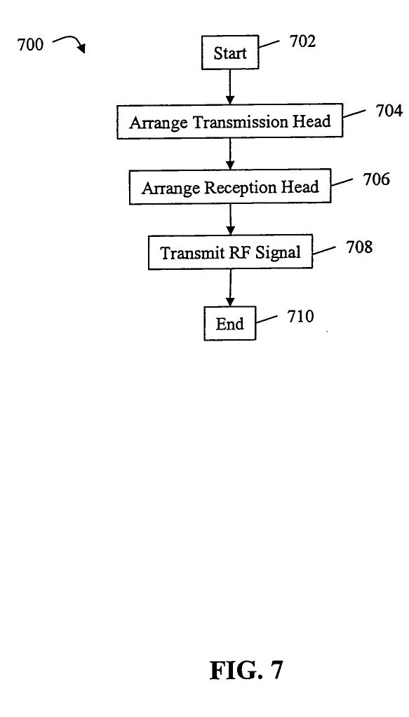

FIG. 7 illustrates a high level exemplary methodology of for inducing hyperthermia in a target area 700. The methodology begins at block 702. At block 704 the transmission head is arranged. Arrangement of the transmission head is accomplished by, for example, placing the transmission head proximate to and on one side of the target area. At block 706 the reception head is arranged. Arrangement of the reception head is similarly accomplished by, for example, placing the reception head proximate to and on the other side of the target area so that an RF signal transmitted via the transmission head to the reception head will pass through the target area. At block 708 the RF signal is transmitted from the transmission head to the reception head. The RF signal passes through and warms cells in the target area. The methodology ends at block 710 and may be ended after a predetermined time interval and/in response to a determination that a desired heating has been achieved.

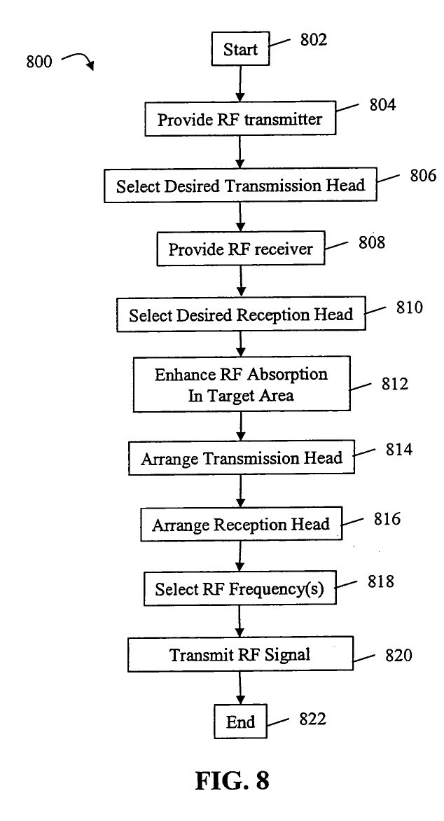

FIG. 8 illustrates an exemplary methodology for inducing hyperthermia in a target area 800. The methodology begins at block 802. At block 804 an RF transmitter is provided. The RF transmitter may be any type of RF transmitter allowing the RF frequency to be changed or selected. Preferably RF transmitter is a variable frequency RF transmitter. Optionally, the RF transmitter is also multi-frequency transmitter capable of providing multiple-frequency RF signals. Still yet, optionally the RF transmitter is capable of transmitting RF signals with variable amplitudes or pulsed amplitudes.

Preferably, a variety of different shapes and sizes of transmission and reception heads are provided. The transmission head is selected at block 806. The selection of the transmission head may be based in part on the type of RF transmitter provided. Other factors, such as, for example, the depth, size and shape of the general target area, or specific target area to be treated, and the number of frequencies transmitted may also be used in determining the selection of the transmission head.

The RF receiver is provided at block 808. The RF receiver may be tuned to the frequency(s) of the RF transmitter. At block 810, the desired reception head is selected. Similarly to the selection of the transmission head, the reception head is preferably selected to fit the desired characteristics of the particular application. For example, a reception head with a small cross section can be selected to concentrate the RF signal on a specific target area. Various sizes and shapes of the reception heads allow for optimal concentration of the RF signal in the desired target area.

The RF absorption in the target area is enhanced at block 812. The RF absorption rate may be enhanced by, for example, injecting an aqueous solution, and preferably an aqueous solution containing suspended particles of an electrically conductive material. Optionally, the RF absorption in the target area is enhanced by exposing the target cells to antibodies bound to an RF absorption enhancer as discussed above.

Arrangement of the transmission head and reception head are performed at blocks 814 and 816 respectfully. The transmission head and reception heads are arranged proximate to and on either side of the target area. The transmission head and reception heads are insulated from the target area. Preferably the heads are insulated from the target area by means of an air gap. Optionally, the heads are insulated from the target area by means of an insulating material. The RF frequency(s) are selected at block 818 and the RF signal is transmitted at block 820. In addition to selecting the desired RF frequency(s) at block 818, preferably, the transmission time or duration is also selected. The duration time is set to, for example, a specified length of time, or set to raise the temperature of at least a portion of the target area to a desired temperature/temperature range, such as, for example to between 106° and 107°, or set to a desired change in temperature. In addition, optionally, other modifications of the RF signal are selected at this time, such as, for example, amplitude, pulsed amplitude, an on/off pulse rate of the RF signal, a variable RF signal where the frequency of the RF signal varies over a set time period or in relation to set temperatures, ranges or changes in temperatures. The methodology ends at block 822 and may be ended after a predetermined time interval and/in response to a determination that a desired heating has been achieved.

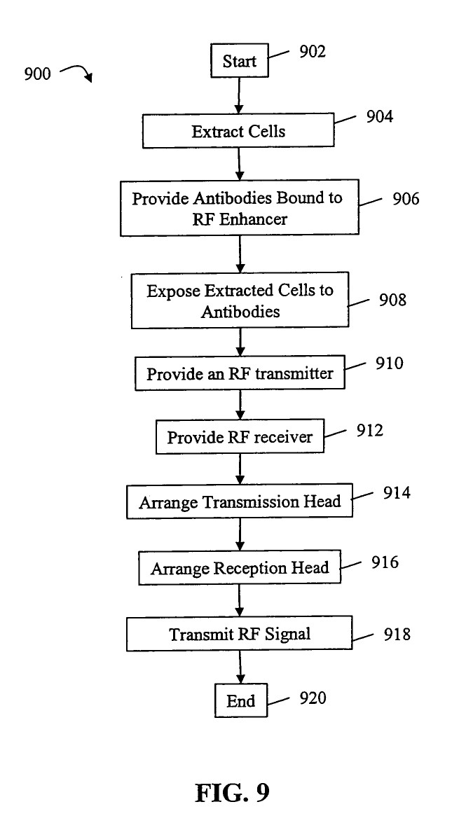

FIG. 9 illustrates an exemplary in-vitro methodology of inducing hyperthermia in target cells 900. The exemplary in-vitro methodology 900 begins at block 902. At block 904, cells to be treated are extracted from a patient and placed in a vessel. The removed cells include at least one or more target cells and are extracted by any method, such as for example, with a needle and syringe. At block 906 antibodies bound with RF enhancers are provided and exposed to the extracted cells. The antibodies bound with RF enhancers attach to one or more of the target cells that are contained within the larger set of extracted cells.

An RF transmitter and RF receiver are provided at blocks 910 and 912 respectively. The transmission head is arranged proximate to and on one side of the target cells in the vessel at block 916. At block 918 the reception head is arranged proximate to and on the other side of the target cells. An RF signal is transmitted at block 918 to increase the temperature of the target cells to, for example, to between 106° and 107°.

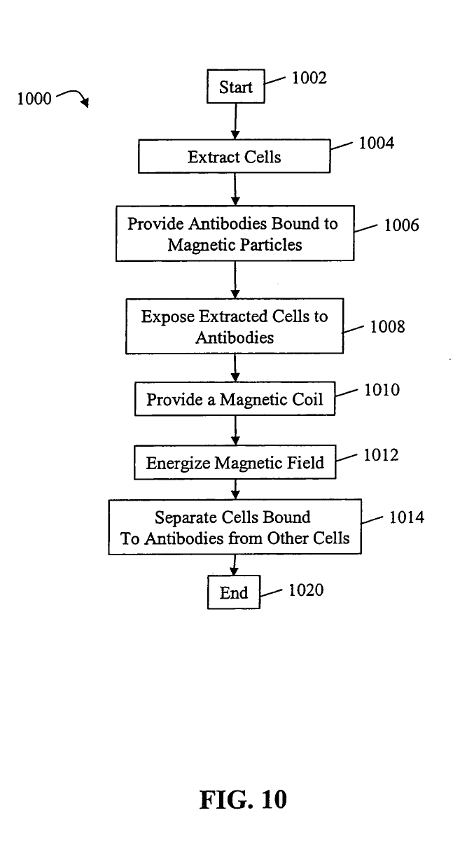

Finally, FIG. 10 illustrates an exemplary in-vitro methodology of separating cells 1000. The exemplary in-vitro methodology begins at block 1002. At block 1004, cells to be treated are extracted from a patient and placed in a vessel. The extracted cells include at least one or more target cells and are extracted by any method, such as for example, with a needle and syringe. At block 1006 antibodies bound with magnetic particles are provided and exposed to the extracted cells. The antibodies bound with magnetic particles attach to one or more of the target cells that are contained within the larger set of extracted cells. A magnetic coil is provided at block 1010 and energized at block 1012. The target cells that are bound to the antibodies are attracted by the magnetic field. The target cells bound to the antibodies are then separated from the other cells. The target cells can be separated by skimming the one or more target cells from the remaining cells, or retaining the one or more target cells in one area of the vessel and removing the other cells. The methodology ends at block 1020 after one or more of the target cells are separated from the other cells.

While the present invention has been illustrated by the description of embodiments thereof, and while the embodiments have been described in some detail, it is not the intention of the applicant to restrict or in any way limit the scope of the appended claims to such detail. Additional advantages and modifications will readily appear to those skilled in the art. For example, modulating the RF signal with another signal, such as, for example, a square wave (e.g. a 300-400 Hz square wave). Modulating the RF signal with a square wave stimulates the tissue and enhances heating. Another example includes total body induced hyperthermia to treat the patient's entire body. In this example, the transmission and reception heads are as large as the patient and hyperthermia is induced in the entire body. Cooling the blood may be required to prevent overheating and can be accomplished in any manner. Therefore, the invention in its broader aspects is not limited to the specific details, representative apparatus and methods, and illustrative examples shown and described. Accordingly, departures may be made from such details without departing from the spirit or scope of the applicant's general inventive concept.

1. A non-invasive RF transceiver system for inducing hyperthermia in at least a portion of a target area, comprising:
(a) an RF transmitter having an RF generator in circuit communication with a transmission head, the RF generator generating an RF signal at a frequency for transmission via the transmission head; and
(b) an RF receiver having a resonant circuit in circuit communication with a reception head, the resonant circuit being tuned to cause at least a portion of the reception head to be resonant at the frequency of the RF signal transmitted via the transmission head so as to receive the RF signal transmitted via the transmission head; and
(c) wherein the transmission and reception heads are arranged proximate to and on either side of the target area so that the RF signal transmitted via the transmission head to the reception head passes through and warms at least a portion of the target area with neither of the heads being in direct contact with the portion of the target area being warmed.

2. The non-invasive RF system for inducing hyperthermia in at least a portion of the target area according to claim 1 wherein the transmission head to the reception head each comprise a plate of electrically conductive material.

3. The non-invasive RF system for inducing hyperthermia in at least a portion of the target area according to claim 1 wherein the resonant circuit is tuned to the frequency of the RF signal generated by the RF generator.

4. The non-invasive RF system for inducing hyperthermia in at least a portion of the target area according to claim 1 wherein the transmission and reception heads are insulated from the target area with sheets of electrically insulating material.

5. The non-invasive RF system for inducing hyperthermia in at least a portion of the target area according to claim 1 wherein the reception head is positioned relative to a specific target portion of the target area in order to concentrate the RF signal through the specific target portion of the target area to warm at least the specific target portion of the target area.

6. The non-invasive RF system for inducing hyperthermia in at least a portion of the target area according to claim 5 wherein the specific target portion of the target area has been injected with an RF absorption enhancer to increase the warming of the specific target portion of the target area by the RF signal.

7. The non-invasive RF system for inducing hyperthermia in at least a portion of the target area according to claim 5 wherein the specific target portion of the target area has been injected with an aqueous solution to increase the warming of the specific target portion of the target area by the RF signal.

8. The non-invasive RF system for inducing hyperthermia in at least a portion of the target area according to claim 5 wherein the specific target portion of the target area has been injected with a saline solution to increase the warming of the specific target portion of the target area by the RF signal.

9. The non-invasive RF system for inducing hyperthermia in at least a portion of the target area according to claim 5 wherein the specific target portion of the target area has been injected with an aqueous suspension of particles of an electrically conductive material to increase the warming of the specific target portion of the target area by the RF signal.

10. The non-invasive RF system for inducing hyperthermia in at least a portion of the target area according to claim 9 wherein the electrically conductive material comprises at least one metal.

11. The non-invasive RF system for inducing hyperthermia in at least a portion of the target area according to claim 5 wherein the specific target portion of the target area has been injected with an aqueous suspension of iron-containing particles to increase the warming of the specific target portion of the target area by the RF signal.

12. The non-invasive RF system for inducing hyperthermia in at least a portion of the target area according to claim 1 wherein the reception head is smaller in cross section than the transmission head and positioned relative to a specific target portion of the target area in order to concentrate the RF signal through the specific target portion of the target area to warm at least the specific target portion of the target area.

13. The non-invasive RF system for inducing hyperthermia in at least a portion of the target area according to claim 12 wherein the reception head is approximately the same shape in cross section as the specific target portion of the target area.

14. A method of inducing hyperthermia in at least a portion of the target area of a patient's body part, comprising the steps of:
(a) providing an RF transmitter having an RF generator in circuit communication with a transmission head, the RF generator capable of generating an RF signal at a frequency for transmission via the transmission head;
(b) providing an RF receiver having a resonant circuit in circuit communication with a reception head, the resonant circuit being tuned to cause at least a portion of the reception head to be resonant at the frequency of the RF signal transmitted via the transmission head so as to receive the RF signal transmitted via the transmission head;
(c) arranging the transmission and reception heads proximate to and on either side of the body part containing the target area in such a manner that the RF signal transmitted via the transmission head to the reception head passes through and warms at least a portion of the target area with neither of the heads being in direct contact with the portion of the target area being warmed;
(d) insulating the transmission and reception heads from the body part;
(e) transmitting the RF signal at the frequency via the transmission head to the reception head, thereby warming the portion of the target area of the body part.

15. The method of inducing hyperthermia in at least a portion of the target area of a patient's body part according to claim 14 wherein step (b), providing a resonant circuit in circuit communication with a reception head, comprises selecting the reception head from a plurality of heads of different sizes and placing the selected head in circuit communication with the resonant circuit.

16. The method of inducing hyperthermia in at least a portion of the target area of a patient's body part according to claim 14 wherein step (c), arranging the transmission and reception heads proximate to and on either side of the body part, comprises positioning the reception head relative to a specific target portion of the target area in order to concentrate the RF signal through the specific target portion of the target area to warm at least the specific target portion of the target area.

17. The method of inducing hyperthermia in at least a portion of the target area of a patient's body part according to claim 16 wherein step (b), providing a resonant circuit in circuit communication with a reception head, comprises selecting the reception head from a plurality of heads and placing the selected reception head in circuit communication with the resonant circuit, and wherein the selected reception head is selected in accordance with the shape and size of the specific target portion of the target area.

18. The method of inducing hyperthermia in at least a portion of the target area of a patient's body part according to claim 14 wherein step (d), insulating the transmission and reception heads from the body part, comprises leaving an air gap between the transmission head and the skin of the body part and leaving an air gap between the reception head and the skin of the body part.

19. The method of inducing hyperthermia in at least a portion of the target area of a patient's body part according to claim 14 wherein step (d), insulating the transmission and reception heads from the body part, comprises inserting electrically insulating material between the transmission head and the body part and inserting a sheet of electrically insulating material between the reception head and the body part.

20. The method of inducing hyperthermia in at least a portion of the target area of a patient's body part according to claim 16 wherein, prior to performing step (e), transmitting the RF signal at the frequency via the transmission head to the reception head, further performing the step of injecting the specific target portion of the target area with an RF absorption enhancer to increase the warming of the specific target portion of the target area by the RF signal.

21. The method of inducing hyperthermia in at least a portion of the target area of a patient's body part according to claim 16 wherein, prior to performing step (e), transmitting the RF signal at the frequency via the transmission head to the reception head, further performing the step of injecting the specific target portion of the target area with an aqueous solution to increase the warming of the specific target portion of the target area by the RF signal.

22. The method of inducing hyperthermia in at least a portion of the target area of a patient's body part according to claim 16 wherein, prior to performing step (e), transmitting the RF signal at the frequency via the transmission head to the reception head, further performing the step of injecting the specific target portion of the target area with a saline solution to increase the warming of the specific target portion of the target area by the RF signal.

23. The method of inducing hyperthermia in at least a portion of the target area of a patient's body part according to claim 16 wherein, prior to performing step (e), transmitting the RF signal at the frequency via the transmission head to the reception head, further performing the step of injecting the specific target portion of the target area with an aqueous suspension of particles of an electrically conductive material to increase the warming of the specific target portion of the target area by the RF signal.

24. The method of inducing hyperthermia in at least a portion of the target area of a patient's body part according to claim 23 wherein the electrically conductive material comprises at least one metal.

25. The method of inducing hyperthermia in at least a portion of the target area of a patient's body part according to claim 16 wherein, prior to performing step (e), transmitting the RF signal at the frequency via the transmission head to the reception head, further performing the step of injecting the specific target portion of the target area with an aqueous suspension of iron-containing particles to increase the warming of the specific target portion of the target area by the RF signal.

26. The method of inducing hyperthermia in at least a portion of the target area of a patient's body part according to claim 16 wherein, prior to performing step (e), transmitting the RF signal at the frequency via the transmission head to the reception head, further performing the step of injecting the specific target portion of the target area with particles that increase the warming of the specific target portion of the target area by the RF signal.

27. The method of inducing hyperthermia in at least a portion of the target area of a patient's body part according to claim 16 wherein, prior to performing step (e), transmitting the RF signal at the frequency via the transmission head to the reception head, further performing the step of injecting the patient with antibodies bound to an RF absorption enhancer, wherein the antibodies bind to at least one type of cells within the specific target portion of the target area, to thereby increase the warming of the type of cells within the specific target portion of the target area by the RF signal.

28. A method of inducing hyperthermia in at least a specific target portion of the target area of a patient's body part, comprising the steps of:
(a) providing an RF transmitter having an RF generator in circuit communication with a transmission head, the RF generator capable of generating an RF signal at a frequency for transmission via the transmission head;
(b) providing an RF receiver having a resonant circuit in circuit communication with a reception head, the resonant circuit being tuned to cause at least a portion of the reception head to be resonant at the frequency of the RF signal transmitted via the transmission head so as to receive the RF signal transmitted via the transmission head, the reception head being selected from a plurality of heads having a shape and size selected in accordance with the shape and size of the specific target portion of the target area and placed in circuit communication with the resonant circuit;
(c) arranging the transmission and reception heads proximate to and on either side of the body part containing the specific target portion of the target area in such a manner that the RF signal transmitted via the transmission head to the reception head passes through and warms at least the specific target portion of the target area with neither of the heads being in contact with the body part, the reception head being positioned relative to the specific target portion of the target area in order to concentrate the RF signal through the specific target portion of the target area to warm at least the specific target portion of the target area;
(d) insulating the transmission and reception heads from the body part;
(e) providing antibodies bound to an RF absorption enhancer, wherein the antibodies bind to at least one type of cells within the specific target portion of the target area, to thereby increase the warming of the type of cells within the specific target portion of the target area by the RF signal;
(f) injecting the antibodies bound to the RF absorption enhancer into the patient;
(g) waiting for the antibodies bound to the RF absorption enhancer to bind to some of the at least one type of cells within the specific target portion of the target area; and
(h) transmitting the RF signal at the frequency via the transmission head to the reception head, thereby warming the specific target portion of the target area of the body part.

29. A method of inducing hyperthermia in at least target cells of a patient, comprising the steps of:
(a) providing an RF transmitter having an RF generator in circuit communication with a transmission head, the RF generator capable of generating an RF signal at a frequency for transmission via the transmission head;
(b) providing an RF receiver having a resonant circuit in circuit communication with a reception head, the resonant circuit being tuned to cause at least a portion of the reception head to be resonant at the frequency of the RF signal transmitted via the transmission head so as to receive the RF signal transmitted via the transmission head;
(c) providing antibodies bound to an RF absorption enhancer, wherein the antibodies bind to the target cells to thereby increase the warming of the target cells by interaction between the RF signal and the RF absorption enhancer;
(d) injecting the antibodies bound to the RF absorption enhancer into the patient;
(e) waiting for the antibodies bound to the RF absorption enhancer to bind to some of the target cells; and
(f) arranging the transmission and reception heads proximate to and on either side of at least one body part of the patient containing the target cells in such a manner that the RF signal transmitted via the transmission head to the reception head passes through and warms at least the target cells with neither of the heads being in direct contact with the body part;
(g) insulating the transmission and reception heads from the body part; and
(h) transmitting the RF signal at the frequency via the transmission head to the reception head, thereby warming the target cells.

30. A method of inducing hyperthermia in at least target cells of a patient, comprising the steps of:
(a) extracting at least the target cells from the patient;
(b) providing an RF transmitter having an RF generator in circuit communication with a transmission head, the RF generator capable of generating an RF signal at a frequency for transmission via the transmission head;
(c) providing an RF receiver having a resonant circuit in circuit communication with a reception head, the resonant circuit being tuned to cause at least a portion of the reception head to be resonant at the frequency of the RF signal transmitted via the transmission head so as to receive the RF signal transmitted via the transmission head;
(d) providing antibodies bound to an RF absorption enhancer, wherein the antibodies bind to the target cells to thereby increase the warming of the target cells by interaction between the RF signal and the RF absorption enhancer;
(e) exposing the target cells to the antibodies bound to the RF absorption enhancer;
(f) waiting for the antibodies bound to the RF absorption enhancer to bind to some of the target cells; and
(g) arranging the transmission and reception heads proximate to and on either side of a vessel containing at least the target cells in such a manner that the RF signal transmitted via the transmission head to the reception head passes through and warms at least the target cells; and
(h) transmitting the RF signal at the frequency via the transmission head to the reception head, thereby warming the target cells.

31. The method of inducing hyperthermia in at least target cells of a patient according to claim 30 wherein step (a), extracting at least the target cells from the patient, comprises the steps of:
(a) removing from the patient material containing at least the target cells;
(b) providing antibodies bound to a magnetic material, wherein the antibodies bind to the target cells;
(c) exposing the target cells to the antibodies bound to the magnetic material;
(d) waiting for the antibodies bound to the magnetic material to bind to some of the target cells; and
(e) providing a magnetic field to attract some of the target cells in such a manner to attract and thereby separate the target cells.

32. The method of inducing hyperthermia in at least target cells of a patient according to claim 31 wherein step (c), exposing the target cells to the antibodies bound to the magnetic material, is performed after step (a), removing from the patient material containing at least the target cells.

33. The method of inducing hyperthermia in at least target cells of a patient according to claim 31 wherein step (c), exposing the target cells to the antibodies bound to the magnetic material, is performed before step (a), removing from the patient material containing at least the target cells.

34. A method of separating target cells of a patient, comprising the steps of:
(a) removing from the patient material containing at least the target cells;
(b) providing antibodies bound to a magnetic material, wherein the antibodies bind to the target cells;
(c) exposing the target cells to the antibodies bound to the magnetic material;
(d) waiting for the antibodies bound to the magnetic material to bind to some of the target cells; and
(e) providing a magnetic field to attract some of the target cells in such a manner to attract and thereby separate the target cells.

35. The method of separating target cells of a patient according to claim 31 wherein the target cells are in the patient's blood and further comprising the step of placing the patient's blood in a vessel, and further wherein step (e), providing a magnetic field to attract some of the target cells in such a manner to attract and thereby separate the target cells, comprises the step of generating a magnetic field aligned with respect to the vessel to attract and thereby separate the target cells from other blood components.

36. A method of inducing hyperthermia in at least a specific target portion of the target area of a patient's body part, comprising the steps of:
(a) providing an RF transmitter having an RF generator in circuit communication with a transmission head, the RF generator capable of generating an RF signal at a frequency for transmission via the transmission head;
(b) providing an RF receiver having a resonant circuit in circuit communication with a reception head, the resonant circuit being tuned to cause at least a portion of the reception head to be resonant at the frequency of the RF signal transmitted via the transmission head so as to receive the RF signal transmitted via the transmission head, the reception head comprising an electrical conductor for insertion into the patient in or near the specific target portion of the target area;
(c) inserting the electrical conductor into the patient in or near the specific target portion of the target area;
(d) arranging the transmission head proximate to the body part containing the specific target portion of the target area in such a manner that the RF signal transmitted via the transmission head to the reception head passes through at least a portion of and warms at least a portion of the specific target portion of the target area with the transmission head being insulated from contact with the body part;
(e) insulating the transmission head from the body part; and
(f) transmitting the RF signal at the frequency via the transmission head to the reception head, thereby warming at least a portion of the specific target portion of the target area of the body part.

37. The method of inducing hyperthermia in at least a specific target portion of the target area of a patient's body part according to claim 36, wherein prior to transmitting the RF signal, further comprising the steps of:
(a) providing antibodies bound to an RF absorption enhancer, wherein the antibodies bind to at least one type of cells within the specific target portion of the target area, to thereby increase the warming of the type of cells within the specific target portion of the target area by the RF signal;
(b) injecting the antibodies bound to the RF absorption enhancer into the patient; and
(c) waiting for the antibodies bound to the RF absorption enhancer to bind to some of the at least one type of cells within the specific target portion of the target area.

38. The method of inducing hyperthermia in at least a specific target portion of the target area of a patient's body part according to claim 36, wherein prior to transmitting the RF signal, further comprising the steps of:
(a) providing antibodies bound to an electrical conductor, wherein the antibodies bind to at least one type of cells within the specific target portion of the target area, to thereby permit at least a portion specific target portion of the target area to help function as the reception head;
(b) injecting the antibodies bound to the electrical conductor into the patient; and
(c) waiting for the antibodies bound to the electrical conductor to bind to some of the at least one type of cells within the specific target portion of the target area.

39. The non-invasive RF system for inducing hyperthermia according to claim 1 wherein:
(a) the RF generator and the transmission head are configured and cooperate to permit the RF generator to generate and the transmission head to transmit a first RF signal at a first frequency and a second RF signal at a second frequency different than the first frequency; and
(b) the resonant circuit and the reception head are configured and cooperate to permit the reception head to receive the first RF signal at the first frequency and the second RF signal at the second frequency.

40. The non-invasive RF system for inducing hyperthermia according to claim 39 wherein the RF generator and the resonant circuit cooperate to alternate between the first frequency and the second frequency.

41. The non-invasive RF system for inducing hyperthermia according to claim 1 wherein:
(a) the transmission head comprises at least first and second portions that are electrically insulated from each other to permit the first portion of the transmission head to transmit a first RF signal at a first frequency to further permit the second portion of the transmission head to transmit a second RF signal at a second frequency different than the first frequency;
(b) the RF generator is in circuit communication with the first and second portions of the transmission head;
(c) the reception head comprises at least first and second portions that are electrically insulated from each other to permit the first portion of the reception head to receive the first RF signal at the first frequency to further permit the second portion of the reception head to receive the second RF signal at the second frequency;
(d) the resonant circuit comprises at least first and second resonant circuit portions, each in circuit communication with one of the first and second portions of the reception head, the first and second resonant circuit portions being tuned to cause the first and second portions of the reception head to be resonant at the respective frequency of the respective RF signal transmitted via the respective portion of the transmission head so as to receive the respective RF signal transmitted via the respective portion of the transmission head; and
(e) the RF generator generates the first RF signal at the first frequency for transmission via the first portion of the transmission head and generates the second RF signal at the second frequency for transmission via the second portion of the transmission head.

42. The non-invasive RF system for inducing hyperthermia according to claim 1 wherein the transmission and reception heads each comprise first and second portions that are electrically insulated from each other.

43. The non-invasive RF system for inducing hyperthermia according to claim 1 wherein the transmission and reception heads are bumpy.

44. The non-invasive RF system for inducing hyperthermia according to claim 1 wherein the transmission and reception heads each have a non-uniform thickness.

45. The non-invasive RF system for inducing hyperthermia according to claim 1 wherein the transmission and reception heads are dimpled.

46. The non-invasive RF system for inducing hyperthermia according to claim 1 wherein the amplitude of the RF signal is substantially constant.

47. The non-invasive RF system for inducing hyperthermia according to claim 1 wherein the amplitude of the RF signal is varied.

48. The non-invasive RF system for inducing hyperthermia according to claim 1 wherein the amplitude of the RF signal is pulsed.

49. The non-invasive RF system for inducing hyperthermia according to claim 1 further comprising the step of cooling the patient's blood in a device external to the patient.

50. An RF system for inducing hyperthermia in a target area comprising:
(a) an RF generator;
(b) a transmission head in circuit communication with the RF generator for transmitting at least one radio frequency; and
(c) a resonant circuit for receiving the radio frequency, wherein the resonant circuit is attached to cells in the target area and the cells in the target area are resonant at the transmitted radio frequency.

51. The RF system for inducing hyperthermia of claim 50 wherein the resonant circuit is attached to the cells in the target area via a needle.

52. The RF system for inducing hyperthermia of claim 51 wherein the needle comprises one or more extension members.

53. The RF system for inducing hyperthermia of claim 50 wherein the resonant circuit is attached to the cells in the target area by a carrier mechanism.

54. The RF system for inducing hyperthermia of claim 53 wherein the carrier mechanism is one or more antibodies.

55. A method of inducing hyperthermia in cells in a target area comprising:
(a) providing one or more resonant circuits;
(b) attaching the one or more resonant circuits to cells in the target area;
(c) providing a transmission head for transmitting the one or more radio frequencies;
(d) positioning the transmission head to transmit the one or more radio frequencies toward the one or more resonant circuits;
(e) transmitting the one or more radio frequencies toward the one or more resonant circuits; and
(f) causing the cells to be resonant while transmitting the one or more radio frequencies.

56. The method of inducing hyperthermia in cells in the target area of claim 55 wherein the step of attaching the one or more resonant circuits to the cells in the target area comprises inserting a needle into the target area.

57. The method of inducing hyperthermia in cells in the target area of claim 55 wherein attaching the one or more resonant circuits to the cells in the target area comprises attaching the one or more resonant circuits to one or more carrier mechanisms.

58. The method of inducing hyperthermia in cells in the target area of claim 57 wherein the one or more carrier mechanisms comprises one or more antibodies.
